(19) United States
(12) Patent Application Publication
(10) Pub. No.: US 2010/0150935 A1
(43) Pub. Date: Jun. 17, 2010

(54) USE OF AN ANTI-CXCR4 ANTIBODY IN THE TREATMENT OF CANCER

(75) Inventor: Christine Klinguer-Hamour, Groisy (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE MEDICAMENT, Boulogne (FR)

(21) Appl. No.: 12/451,025

(22) PCT Filed: Apr. 18, 2008
(86) PCT No.: PCT/FR2008/000553

§ 371 Date: Oct. 22, 2009

(30) Foreign Application Priority Data
Apr. 23, 2007 (FR) 0702977

(51) Int. Cl. A61K 39/395; A61P 35/00

(52) U.S. Cl. 424/155.1

(57) ABSTRACT

The present invention relates to the use of at least one antibody, or a functional fragment thereof, which is capable of binding to the protein CXCR4 and thereby inhibiting tumour growth, in the preparation of a medicament intended for the treatment of cancer.

The invention is also directed to a composition for the treatment of cancer, comprising, as active ingredient, at least one anti-CXCR4 antibody, or a functional fragment thereof, which is capable of binding to the protein CXCR4 and/or of inhibiting its angiogenic and/or proliferative activity. More especially, said antibodies consist of the antibody MAB173.

The present invention relates to a new use of anti-CXCR4 antibodies capable of inhibiting tumour growth, said antibodies being especially monoclonal of murine origin, chimaeric and humanised. According to a particular aspect, the invention relates to the use of those antibodies, or of functional fragments thereof, as a medicament for the prophylactic and/or therapeutic treatment of cancers. Finally, the invention includes products and/or compositions comprising such antibodies in association, for example, with anti-cancer agents and/or antibodies or conjugated with toxins, and use thereof in the prevention and/or treatment of certain cancers.

The CXCR4 receptor is a membrane receptor coupled to G proteins of the chemokine receptor family.

Two isoforms of the CXCR4 receptor exist, the one consisting of 352 amino acids and the other of 360. The residue Asn11 is glycosylated, the residue Tyr21 is modified by the addition of a sulfate group and there is a disulfide bridge between Cys 109 and Cys 186 on the extracellular portion of the receptor.

The receptor is expressed by a certain number of healthy tissues, including endothelial cells, lymphocytes, macrophages, dendritic cells, “Natural Killer” cells and, to a minor degree, in the heart, colon, liver, kidneys and brain. The CXCR4 receptor is overexpressed in a large number of cancers, including cancer of the colon, breast, prostate, lung (small-cell and non-small-cell), ovary, pancreas, kidney, brain and certain lymphomas. CXCR4 expression is increased in metastatic cells compared to cancer cells from the primary tumour.

The only known ligand of this receptor is “Stromal-Derived Factor-1 (SDF-1)”, or CXCL12, which is secreted in large amounts by cells of the lymphatic ganglia, bone marrow, liver and lung and, in small amounts, in the kidneys, brain and skin.

The CXCR4/SDF-1 axis plays an important part in cancer because it is directly involved in the phenomena of cell migration and the formation of metastases. In fact, cancer cells express the CXCR4 receptor; they migrate and enter the blood stream. They come to a halt in organs that produce large amounts of SDF-1, where they multiply and form metastases (Murphy P M., 2001).

It has also been shown that the CXCR4/SDF-1 axis plays a part in angiogenesis. More specifically, it has been clearly demonstrated in vitro that the CXCR4 receptor and its ligand SDF-1 promote angiogenesis by stimulating the expression of VEGF-A, which in its turn increases the expression of CXCR4/SDF-1 (Bachelder R. E. et al., 2002).

The value of targeting metastases by interfering with the CXCR4 receptor has especially been demonstrated in vivo by using a monoclonal antibody directed to CXCR4 (Muller A. et al., 2001). In an orthotopic model of breast cancer (cell line MDA-MB231) in the SCID mouse, a monoclonal antibody directed to CXCR4 is indeed capable of significantly reducing the number of metastases in the ganglia. A complementary study (Phillips R. J et al. 2003) has likewise shown the role of the SDF-1/CXCR4 axis in the formation of metastases in a model of lung cancer (A549).

Furthermore, the macrophages associated with tumours are key components of the inflammatory circuits for tumour growth. The chemokines, including SDF-1, take part in the recruitment of macrophages in tumours. Monoclonal antibodies capable of recognising the CXCR4 receptor might also be able to reduce the recruitment of macrophages in tumours, thereby limiting their effect on tumour growth.

According to a general aspect, the present invention is directed to use of an antibody, or a functional fragment thereof, which is capable of specifically binding to the protein CXCR4, in the treatment of cancer.

The functional fragments of antibodies according to the invention consist, for example, of Fv, scFv (sc standing for single chain), Fab, F(ab′)₂, Fab′ or scFv-Fc fragments or diabodies, or any fragment whose half-life may have been extended by chemical modification, e.g. addition of poly(alkylene)glycol such as poly(ethylene)glycol (“PEGylation”) (the PEGylated fragments being referred to as Fv-PEG, scFv-PEG, Fab-PEG, F(ab′)₂-PEG or Fab′-PEG) (“PEG” from the designation Poly(Ethylene)Glycol), or by incorporation in a liposome, microspheres or PLGA, said fragments being capable of generally exerting activity, even partial, of the antibody from which they are derived.

Preferably, said functional fragments will be composed of or will comprise a partial sequence of the variable heavy or light chain of the antibody from which they are derived, said partial sequence being sufficient to retain the same binding specificity as the antibody from which it is derived and an adequate affinity, preferably equal to at least 1/100th, more preferably at least 1/10th, of that of the antibody from which it is derived.

Such a functional fragment will comprise at least 5 consecutive amino acids, preferably 10, 15, 25, 50 or 100 consecutive amino acids, from the sequence of the antibody from which it is derived.

Preferably, these functional fragments will be fragments of Fv, scFv, Fab, F(ab′)₂, F(ab′), scFv-Fc type, or diabodies, which generally have the same binding specificity as the antibody from which they are derived. According to the present invention, fragments of antibodies of the invention can be obtained starting from antibodies as described hereinbefore by methods such as digestion using enzymes such as pepsin or papain and/or by cleavage of the disulfide bridges by means of chemical reduction. Otherwise, the antibody fragments included in the present invention can be obtained by genetic recombination techniques that are likewise well-known to the person skilled in the art or by peptide synthesis by means of, for example, automatic peptide synthesisers such as those supplied by the company Applied.

According to an aspect of the invention, the antibody used consists of a murine monoclonal antibody.

Antibodies according to the present invention also include chimaeric or humanised antibodies.

A chimaeric antibody is understood to denote an antibody which contains a natural variable (light chain and heavy chain) region derived from an antibody from a given species in association with the constant light chain and heavy chain regions of an antibody from a heterologous species to said given species.

Chimaeric-type antibodies, or their fragments, used in accordance with the invention can be prepared using genetic recombination techniques. For example, the chimaeric antibody may be produced by cloning a recombinant DNA comprising a promoter and a sequence coding for the variable region of a non-human, especially murine, monoclonal antibody according to the invention and a sequence coding for the constant region of a human antibody. A chimaeric antibody of the invention encoded by such a recombinant gene may be, for example, a mouse-human chimaera, the specificity of that antibody being determined by the variable region derived from murine DNA and its isotype determined by the constant region derived from human DNA. For methods of preparing chimaeric antibodies, reference may be made, for example, to the document Verhoeyn et al. (BioEssays, 8:74, 1988).

A humanised antibody is understood to denote an antibody which contains CDR regions derived from an antibody of non-human origin, the other parts of the antibody molecule being derived from one (or more) human antibody/antibodies. In addition, some of the residues of the segments of the skeleton (referred to as FR) can be modified in order to preserve the binding affinity (Jones et al., Nature, 321:522-525, 1986; Verhoeyen et al., Science, 239:1534-1536, 1988; Riechmann et all, Nature, 332:323-327, 1988).

The humanised antibodies or functional fragments thereof can be prepared by techniques known to the person skilled in the art (such as, for example, those described in the documents Singer et al., J. Immun. 150:2844-2857, 1992; Mountain et al., Biotechnol. Genet. Eng. Rev., 10:1-142, 1992; or Bebbington et al., Bio/Technology, 10:169-175, 1992). Such humanised antibodies are preferred for their use in in vivo prophylactic and/or therapeutic treatment methods. Other humanisation techniques are also known to the person skilled in the art, such as, for example, the technique of “CDR Grafting”, described by PDL, which is the subject-matter of patents EP 0 451 261, EP 0 682 040, EP 0 939 127, EP 0 566 647 or also U.S. Pat. No. 5,530,101, U.S. Pat. No. 6,180,370, U.S. Pat. No. 5,585,089 and U.S. Pat. No. 5,693,761. U.S. Pat. No. 5,639,641 or also U.S. Pat. No. 6,054,297, U.S. Pat. No. 5,886,152 and U.S. Pat. No. 5,877,293 may also be mentioned.

More specifically, the Applicant is putting forward, without wishing to be bound by any such theory, that the use of anti-CXCR4 antibodies in the context of cancer treatment should be of value, not solely due to the fact of inhibition of the metastasis-promoting activity of CXCR4 as is suggested by the prior art, but also and above all due to the fact of action directly in the primary tumour.

Indeed, as can be seen hereinbefore, it is known to the skilled person that blocking the CXCR4/SDF-1 axis by means of an antibody makes it possible to significantly reduce the number of metastases in the ganglia (Muller et al., 2001). Moreover, it is likewise described that although in vivo blocking of that axis significantly reduces metastases it does not alter the size of the primary tumour nor the angiogenic activity within the tumour (Phillips R J et al., 2003).

Consequently, the prior art clearly teaches that use of an anti-CXCR4 antibody, i.e. an antibody capable of interfering with the CXCR4/SDF-1 axis, is solely of value in respect of metastases.

The present invention is innovative in the sense that, for the first time and contrary to the preconceptions of the person skilled in the art, it describes and claims use of a monoclonal anti-CXCR4 antibody which is capable not only of acting in respect of metastases but, above all, of acting directly in respect of the tumour growth of primary tumours.

More specifically, the present invention relates to use of a monoclonal antibody, or a functional fragment thereof, which is capable of specifically binding to the protein CXCR4, in inhibiting in vitro and/or in vivo tumour growth of a primary tumour.

Tumour cell transformation is reflected especially by a loss of control over the cell cycle, insensitivity to apoptosis of DNA repair anomalies. The cancers are then classified according to the type of cell in which the first transformation occurred (lymphomas, carcinomas, sarcomas); that first malignant cell then divides to form the primary tumour. Some primary tumours can progress towards more complete invasion of the body, as a result of the escape of tumour cells derived from that primary tumour: these are then referred to as metastases.

It must accordingly be understood that, within the context of the present invention, the expression “primary tumour” is to be contrasted with “metastases”. The primary tumour constitutes the first stage of development of a cancer whereas metastases constitute a different and subsequent stage into which primary tumours may develop.

A “metastasis” is to be understood then as a secondary site of infection formed following the dissemination of cancer cells from the first site, or primary tumour, by way of the blood or lymphatic system.

The present invention accordingly proposes an alternative to the existing treatments in the sense that it provides early treatment of primary tumours before the latter metastasise.

“Tumour growth” of a primary tumour is to be understood as an increase in the volume of said primary tumour, this increase in volume resulting from various mechanisms, namely vascularisation, apoptosis or also cell proliferation.

“Vascularisation” is to be understood as an increase in vascularisation in the primary tumour by means of angiogenesis and/or vasculogenesis.

Angiogenesis consists of the formation of new vessels (neovascularisation) originating from a pre-existing capillary network as a result of proliferation and migration of endothelial cells, especially in the course of wound healing or development of a cancerous tumour.

Vasculogenesis consists of a process whereby endothelial cells and a primitive plexus are generated by differentiation of precursors of the endothelial cells (angioblasts and haemangioblasts).

“Apoptosis” is to be understood as the process whereby cells trigger their own destruction in response to a signal. It is genetically programmed cell death.

“Cell proliferation” is to be understood as any process involved in an increase in the number of cells. Such processes relate moreover to the cell division that forms part of the cell cycle. More especially, cell proliferation comprises the uncontrolled and excessive division of cells which gives rise to a tumour.

The present invention accordingly describes use of a monoclonal antibody as described hereinbefore, or a functional fragment thereof, which is capable of inhibiting vascularisation, that is to say angiogenesis and/or vasculogenesis, in the primary tumour.

In accordance with a second aspect, the present invention describes use of an antibody as described hereinbefore, or a functional fragment thereof, which is capable of inhibiting cell proliferation of tumour cells forming the primary tumour.

In accordance with a third aspect, the present invention describes use of an antibody as described hereinbefore, or a functional fragment thereof, which is capable of inhibiting vascularisation in the primary tumour and cell proliferation of tumour cells forming said primary tumour.

Finally, in accordance with yet another aspect, the present invention describes use of an antibody as described hereinbefore, or a functional fragment thereof, which in addition is capable of inducing apoptosis of tumour cells forming the primary tumour.

Preferably, the invention principally comprises use of at least one anti-CXCR4 antibody, or a functional fragment thereof, which consists of a monoclonal antibody.

A “monoclonal antibody” is to be understood as an antibody that is derived from a population of substantially homogeneous antibodies. More especially, the individual antibodies of a population are identical except for a few possible mutations which may occur naturally and be present in minimal amounts. In other words, a monoclonal antibody consists of a homogeneous antibody which results from the proliferation of a single cell clone (for example, a hybridoma, a eukaryotic host cell transfected with a DNA molecule coding for the homogeneous antibody, a prokaryotic host cell transfected with a DNA molecule coding for the homogeneous antibody, etc.) and which is generally characterised by heavy chains of one and the same class and sub-class and light chains of a single type. Monoclonal antibodies are highly specific and are directed to a single antigen. In addition, unlike preparations of polyclonal antibodies which customarily comprise different antibodies directed to different determinants, or epitopes, each monoclonal antibody is directed to a single epitope of the antigen.

In accordance with a particular embodiment of the invention, the monoclonal antibody used is selected from the antibodies MAB170 (derived from the clone 12G5), MAB171 (derived from the clone 44708), MAB172 (derived from the clone 44716) and MAB173 (derived from the clone 44717) (http://www.rndsystems.com). In the present description, each antibody may be designated by its name or by the name of the hybridoma from which it is derived. For example, the antibody MAB173 may be interchangeably referred to as, especially in the Examples, 44717 or MAB173 or also Mab173.

Table 1 hereinbelow gives a non-exhaustive listing, for each clone, of the antibodies that are available to the skilled person from R&D Systems.

TABLE 1

| CLONE | MAB (antibody) |
|---|---|
| 12G5 | MAB170 |
| 44708 | MAB171 |
| 44716 | MAB172 |
| 44717 | MAB173 |

More especially, the monoclonal antibody according to the invention consists of the antibody MAB173.

The present invention accordingly describes use of an anti-CXCR4 monoclonal antibody, or a functional fragment thereof, said monoclonal antibody consisting of the antibody MAB173.

The monoclonal antibody MAB173 was produced from a hybridoma resulting from the fusion of a murine myeloma with B cells isolated from a mouse having been inoculated with murine 3T3 cells transfected with the protein hCXCR4. The IgGs were then purified from the ascites fluid by Protein G affinity chromatography (R&D Systems technical data sheet relating to the antibody MAB173).

This antibody is described as specifically recognising the human protein CXCR4 and not recognising the equivalent rat protein; nor does this antibody recognise other chemokine receptors (R&D Systems technical data sheet).

An embodiment of the invention envisages use of an antibody as described hereinbefore in the preparation of a medicament intended for the treatment and/or prevention of cancer.

Preferably, the use of the anti-CXCR4 antibodies in the context of the treatment and/or prevention of cancer is appropriate, very especially, in cancers overexpressing that same CXCR4 receptor.

Such cancers comprise cancers of the colon, breast, prostate, lung (small-cell and non-small-cell), ovary, pancreas, kidney, brain and certain lymphomas.

The present invention accordingly claims use of an antibody as described hereinbefore in the treatment of cancer, said cancer being selected from cancer of the colon, breast, prostate, lung (small-cell and non-small-cell), ovary, pancreas, kidney, brain and certain lymphomas.

The invention relates also to a pharmaceutical composition comprising, as active ingredient, a compound consisting of an antibody, or one of its derivative compounds or functional fragments, to which there is preferably added an excipient and/or a pharmaceutically acceptable carrier.

More especially, the invention is directed to use of an antibody according to the invention in the preparation of a pharmaceutical composition additionally comprising at least one pharmaceutically acceptable carrier.

In the present description, a pharmaceutically acceptable carrier is understood as referring to a compound or combination of compounds included in a pharmaceutical composition which does not give rise to secondary reactions and which, for example, makes it possible to facilitate the administration of the active compound(s), to increase the life and/or efficacy thereof in the body, to increase the solubility thereof in solution or to improve its storage. Such pharmaceutically acceptable carriers are well-known and will be adapted by the person skilled in the art as a function of the nature and mode of administration of the selected active compound(s).

Preferably, those compounds will be administered by a, systemic route, especially by the intravenous route, by the intramuscular, intradermal, intraperitoneal or subcutaneous route, or by the oral route. More preferably, the composition comprising the antibodies according to the invention will be administered on a plurality of occasions staggered over time.

Their optimal modes of administration, dosage regimens and galenic forms can be determined according to criteria generally taken into consideration in establishing a suitable treatment for a patient such as, for example, the age or bodyweight of the patient, the severity of his or her general condition, the tolerability of the treatment and the secondary effects established.

According to the invention there is described a composition for the treatment of cancer, characterised in that it comprises, as active ingredient, at least one anti-CXCR4 antibody, or a functional fragment thereof, which is capable of binding to the protein CXCR4.

According to another aspect of the invention, there is described a composition comprising at least one anti-CXCR4 antibody, or a functional fragment thereof, said at least one antibody being a monoclonal antibody selected from the antibodies MAB170, MAB171, MAB172 and MAB173, preferably the antibody MAB173.

According to a further aspect of the invention, there is claimed a composition which comprises a combination of the antibodies mentioned hereinbefore, or of functional fragments thereof.

As seen hereinbefore, the invention is directed also to use of a composition comprising at least one antibody as described hereinbefore in the treatment of cancer of the colon, breast, prostate, lung (small-cell and non-small-cell), ovary, pancreas, kidney, brain and certain lymphomas.

Of course, the above list is given solely by way of illustration and any cancer must be understood as overexpressing the protein CXCR4 and therefore as being capable of being treated in accordance with the present invention.

Another, complementary embodiment of the invention consists of a composition as described hereinbefore, characterised in that it additionally comprises, as a combination product for simultaneous, separate or time-staggered use, at least one cytotoxic/cytostatic agent and/or cell toxin and/or radioelement.

“Simultaneous use” is understood as the administration of the two compounds of the composition according to the invention contained in one and the same pharmaceutical form.

“Separate use” is understood as the administration, at the same time, of the two compounds of the composition according to the invention contained in separate pharmaceutical forms.

“Time-staggered use” is understood as the successive administration of the two compounds of the composition according to the invention, each contained in a separate pharmaceutical form.

In general manner, the composition according to the invention considerably increases the efficacy of the cancer treatment. In other words, the therapeutic effect of the antibody according to the invention is potentiated in unexpected manner by the administration of a cytotoxic agent. Another major subsequent advantage produced by a composition according to the invention relates to the possibility of using lower effective doses of active ingredient, which makes it possible to avoid or reduce the risks of secondary effects appearing, especially the effect of the cytotoxic agent. Moreover, this composition according to the invention should make it possible to achieve the expected therapeutic effect more rapidly.

“Anti-cancer therapeutic agents” or “cytotoxic agents” are to be understood as substances which, when administered to a patient, treat or prevent the development of the cancer in the patient. By way of non-limiting example of such agents there may be mentioned “alkylating” agents, antimetabolites, anti-tumour antibiotics, mitotic inhibitors, chromatin function inhibitors, anti-angiogenesis agents, anti-oestrogens, anti-androgens or immunomodulators.

Such agents are, for example, mentioned in the VIDAL, on the page devoted to compounds used in oncology and haematology in the column “Cytotoxiques” (English: cytotoxic agents); such cytotoxic compounds mentioned by way of reference to that document are mentioned here as preferred cytotoxic agents.

“Alkylating agents” refer to any substance which is capable of covalently binding to or alkylating any molecule, preferably a nucleic acid (e.g.: DNA), within a cell. As examples of such alkylating agents there may be mentioned nitrogen mustards such as mechlorethamine, chlorambucil, melphalan hydrochloride, pipobroman, prednimustine disodium phosphate or estramustine; oxazaphosphorines such as cyclophosphamide, altretamine, trofosfamide, sulfofosfamide or ifosfamide; aziridines or ethylene-imines such as thiotepa, triethyleneamine or altetramine; nitrosoureas such as carmustine, streptozocin, fotemustine or lomustine; alkyl sulfonates such as busulfan, treosulfan or improsulfan; triazenes such as dacarbazine; and also platinum complexes such as cisplatin, oxaliplatin or carboplatin.

“Antimetabolites” refer to substances which block cell growth and/or cell metabolism by interfering with certain activities, generally DNA synthesis. By way of example of antimetabolites there may be mentioned methotrexate, 5-fluorouracil, floxuridine, 5-fluorodeoxyuridine, capecitabine, cytarabine, fludarabine, cytosine arabinoside, 6-mercaptopurine (6-MP), 6-thioguanine (6-TG), chlorodeoxyadenosine, 5-azacytidine, gemcitabine, cladribine, deoxycoformycin and pentostatin.

“Anti-tumour antibiotics” refer to compounds which can prevent or inhibit the synthesis of DNA, of RNA and/or of proteins. Examples of such anti-tumour antibiotics include doxorubicin, daunorubicin, idarubicin, valrubicin, mitoxantrone, dactinomycin, mithramycin, plicamycin, mitomycin C, bleomycin and procarbazine.

“Mitotic inhibitors” prevent the normal progression of the cell cycle and mitosis. In general, the microtubule inhibitors or “taxoids” such as paclitaxel and docetaxel are capable of inhibiting mitosis. The vinca alkaloids such as vinblastine, vincristine, vindesine and vinorelbine are also capable of inhibiting mitosis.

“Chromatin function inhibitors” or “topoisomerase inhibitors” refer to substances which inhibit the normal function of chromatin remodelling proteins such as topoisomerases I and II. Examples of such inhibitors include, for topoisomerase I, camptothecin and also its derivatives such as irinotecan or topotecan and, for topoisomerase II, etoposide, etiposide phosphate and teniposide.

“Anti-angiogenesis agents” refer to any drug, compound, substance or agent which inhibits the growth of blood vessels. Examples of anti-angiogenesis agents include, without any limitation, razoxin, marimastat, batimastat, prinomastat, tanomastat, ilomastat, CGS-27023A, halofuginone, COL-3, neovastat, BMS-275291, thalidomide, CDC 501, DMXAA, L-651582, squalamine, endostatin, SU5416, SU6668, interferon-alpha, EMD121974, interleukin-12, IM862, angiostatin and vitaxin.

“Anti-oestrogens” or “anti-oestrogen agents” refer to any substance which reduces, antagonises or inhibits the action of oestrogens. Examples of such agents are tamoxifen, toremifene, raloxifene, droloxifene, iodoxyfene, anastrozole, letrozole and exemestane.

“Anti-androgens” or “anti-androgen agents” refer to any substance which reduces, antagonises or inhibits the action of an androgen. Examples of anti-androgens are flutamide, nilutamide, bicalutamide, spironolactone, cyproterone acetate, finasteride and cimitidine.

Immunomodulators are substances which stimulate the immune system. Examples of such immunomodulators include interferons, interleukins such as aldesleukin, OCT-43, denileukin diflitox or interleukin-2, tumour necrosis factors such as tasonermin, or other types of immunomodulators such as lentinan, sizofiran, roquinimex, pidotimod, pegademase, thymopentin, poly I:C, or levamisole in combination with 5-fluorouracil.

For further details, the person skilled in the art will be able to refer to the manual published by the French Association of Teachers of Therapeutic Chemistry entitled “Traité de chimie thérapeutique, Vol. 6, Médicaments antitumoraux et perspectives dans le traitement des cancers, ed. TEC & DOC, 2003”.

In an especially preferred embodiment, said composition in the form of a combination product according to the invention is characterised in that said cytotoxic agent is chemically bound to said antibody for simultaneous use.

In an especially preferred embodiment, said composition according to the invention is characterised in that said cytotoxic/cytostatic agent is selected from spindle inhibitor or stabiliser agents, preferably vinorelbine and/or vinflunine and/or vincristine.

In order to facilitate binding between said cytotoxic agent and said antibody according to the invention, it will be possible, especially, to introduce spacer molecules between the two compounds to be bound, e.g. poly(alkylene)glycols such as polyethyleneglycol, or also amino acids, or, in another embodiment, to use active derivatives of said cytotoxic agents into which there will have been introduced functions capable of reacting with said antibody according to the invention. These binding techniques are well known to the person skilled in the art and will not be elaborated upon in the present description.

According to another aspect, the invention relates to a composition characterised in that one, at least, of said antibodies, or one of their derivative compounds or functional fragments, is conjugated with a cell toxin and/or a radioelement.

Preferably, said toxin or said radioelement is capable of preventing the growth or proliferation of the tumour cell, especially of totally inactivating said tumour cell.

Preference is also given to said toxin being an enterobacterial toxin, especially Pseudomonas exotoxin A.

The radioelements (or radioisotopes) employed in therapy, preferably conjugated with the antibody, are radioisotopes which emit gamma rays, preferably iodine¹³¹, yttrium⁹⁰, gold¹⁹⁹, palladium¹⁰⁰, copper⁶⁷, bismuth²¹⁷ and antimony²¹¹. Radioisotopes which emit beta and alpha rays may also be used in therapy.

A toxin or radioelement conjugated with at least one antibody, or a functional fragment thereof, according to the invention is understood to refer to any means making it possible to bind said toxin or said radioelement to said at least one antibody, especially by covalent binding between the two compounds, with or without introduction of a linking molecule.

Among the agents allowing chemical (covalent), electrostatic or non-covalent linkage of all or some of the conjugate's elements there may be mentioned, very especially, benzoquinone, carbodiimide and, more especially, EDC (1-ethyl-3-[3-dimethylaminopropyl]-carbodiimide hydrochloride), dimaleimide, dithiobis-nitrobenzoic acid (DTNB), N-succinimidyl S-acetyl thioacetate (SATA), agents referred to as “bridging” agents having one or more groups, with one or more phenylazide groups, reacting with ultraviolet (UV) and very preferably N-[-4-(azidosalicylamino)butyl]-3′-(2′-pyridyldithio)propionamide (APDP), N-succinimidyl 3-(2-pyridyldithio)propionate (SPDP) and 6-hydrazino-nicotinamide (HYNIC).

Another form of binding, very especially for radioelements, can consist of using a bifunctional ion chelator.

Among those chelators there may be mentioned the chelators derived from EDTA (ethylenediaminetetraacetic acid) or DTPA (diethylenetriaminepentaacetic acid) that have been developed for binding metals, especially radioactive metals, and immunoglobulins. Accordingly, DTPA and its derivatives can be substituted with different groups on the carbon chain so as to increase the stability and rigidity of the ligand-metal complex (Krejcarek et al. (1977); Brechbiel et al. (1991); Gansow (1991); U.S. Pat. No. 4,831,175).

For example, DTPA (diethylenetriaminepentaacetic acid) and its derivatives, which has long been used very widely in medicine and biology either in its free form or in the form of a complex with a metal ion, has the noteworthy characteristic of forming stable chelates with metal ions and of being bound to proteins of therapeutic or diagnostic interest such as antibodies for the development of radioimmunoconjugates in cancer therapy (Meases et al., (1984); Gansow et al. (1990)).

In accordance with yet another aspect, the composition according to the invention additionally comprises at least one second antibody known for its anti-tumour activity. By way of non-limiting example there may be mentioned the antibodies anti-Her2/neu (Herceptin), anti-EGFR (Erbitux) and also anti-IGF-1R (7C10 or h7C10). Of course, any anti-tumour antibody may be included in the composition according to the invention.

The present invention additionally comprises use of the composition according to the invention in the preparation of a medicament.

The present invention is accordingly directed more especially to use of a composition as described hereinbefore in the preparation of a medicament intended for the treatment of cancer. Among the cancers which may be prevented and/or treated preference is given to cancer of the colon, breast, prostate, lung (small-cell and non-small-cell), ovary, pancreas, kidney, brain and certain lymphomas.

The invention relates also to the use of an antibody according to the invention in the preparation of a medicament intended for the specific targeting of a biologically active compound at cells expressing or overexpressing the CXCR4 receptor.

A biologically active compound is understood herein as referring to any compound capable of modifying, especially inhibiting, the activity of cells, especially their growth, their proliferation, or the transcription or translation of genes.

Other characteristics and advantages of the invention will emerge in the remainder of the description with the Examples and Figures, for which the legends are given hereinbelow.

### LEGENDS FOR FIGURES

FIG. 1 illustrates the study of inhibition, by SDF1 and the antibody 44717, of the binding of [¹²⁵I]SDF1 to membranes of CHO-K1 cells constitutively expressing the human CXCR4 receptor.

FIG. 2 illustrates the study of the binding of [³⁵S]GTPγS to membranes of CHO-K1 cells constitutively expressing the human CXCR4 receptor: influence of SDF1 and of the antibody 44717.

FIG. 3 illustrates the study of the release of intracellular calcium on intact CHO-K1 cells expressing the CXCR4 receptor: influence of SDF1 and of the antibody 44717.

FIG. 4 shows the in vivo anti-tumour activity of the monoclonal antibody 44717 in the U-937 xenograft model.

FIG. 5 shows the inhibition, by the monoclonal antibody 44717, of tumour growth in the MDA-MB-231 xenograft model.

### EXAMPLES

### Example 1

### Test for Inhibition, by the Antibody 44717 (Otherwise Designated MAB173), of the Binding of [¹²⁵I]SDF-1

Method:

CHO-K1 cells constitutively expressing the human CXCR4 receptor are obtained by stable transfection with an expression vector containing the totality of the sequence coding for human CXCR4. These cells are cultured in complete DMEM-Ham's F12 culture medium containing 5% foetal calf serum (FCS) and 500 μg/ml of geneticin. The binding tests are carried out on cell membranes obtained after mechanical scraping of the cells into a buffer [Hepes 20 mM, pH 7.4, NaCl 150 mM] and harvested by centrifugation 10000 g, 15 min. The binding of [¹²⁵I]SDF1 (specific activity: 1500 Ci/mmol) is measured in a homogeneous medium with the aid of SPA beads (scintillation proximity assay—GE Healthcare) in 96-well plates. Briefly, the membranes (30 μg/well) are incubated in the binding buffer [Hepes 20 mM, pH 7.4, CaCl₂ 1 mM, MgCl₂ 5 mM, NaCl 150 mM, BSA 1%] with the compound being studied (SDF1 or antibody 44717), the radioligand (1 nM) and then the SPA-WGA-PVT beads (7.3 mg/well) and incubated for 1 hour at 25° C. After centrifugation [10 min. at 1000 g], the radioactivity is read off in a scintillation counter (TopCount, Perkin Elmer). Non-specific binding of [¹²⁵I]SDF1 is determined in the presence of 10 μM of non-labelled SDF1.

Results:

SDF1 inhibits the binding of [¹²⁵I]SDF1 in dose-dependent manner, with an IC₅₀ value (50% inhibition of the specific binding of [¹²⁵I]SDF1) of 25.2±6.4 nM (n=6) (FIG. 1). Under these test conditions, the antibody 44717 shows maximum inhibition of the binding of [¹²⁵I]SDF1 of 75% at a dose of 1 μM and an IC₅₀ value of 44.8±5.6 nM (n=3) (FIG. 1).

### Example 2

### Test for Modulation, by the Antibody 44717, of the Binding of [³⁵S]GTPγS

This test makes it possible to study the modulation of the activation of heterotrimeric G proteins mediated by CXCR4 and ligands bound thereto.

Method:

CHO-K1 cells constitutively expressing the human CXCR4 receptor are obtained in the same manner as described in Example 1. The binding tests are carried out on cell membranes obtained after mechanical scraping of the cells into a buffer [Hepes 20 mM, pH 7.4, NaCl 150 mM] and harvested by centrifugation 10000 g, 15 min. The binding of [³⁵S]GTPγS (specific activity 1000 Ci/mmol) is measured in a homogeneous medium with the aid of SPA beads (scintillation proximity assay—GE Healthcare) in 96-well plates. Briefly, the membranes (10 μg/well) are incubated in the binding buffer [Hepes 20 mM, GDP 3 μM, MgCl₂ 10 mM, NaCl 100 mM, EDTA 1 mM, pH=7.4] with the compound being studied (SDF1 and/or the antibody 44717), [³⁵S]GTPγS (0.2-0.4 nM) and then the SPA-WGA-PVT beads (7.3 mg/well) and incubated for 1 hour at 25° C. After centrifugation [10 min. at 1000 g] the radioactivity is read off in a scintillation counter (TopCount, Perkin Elmer).

Results:

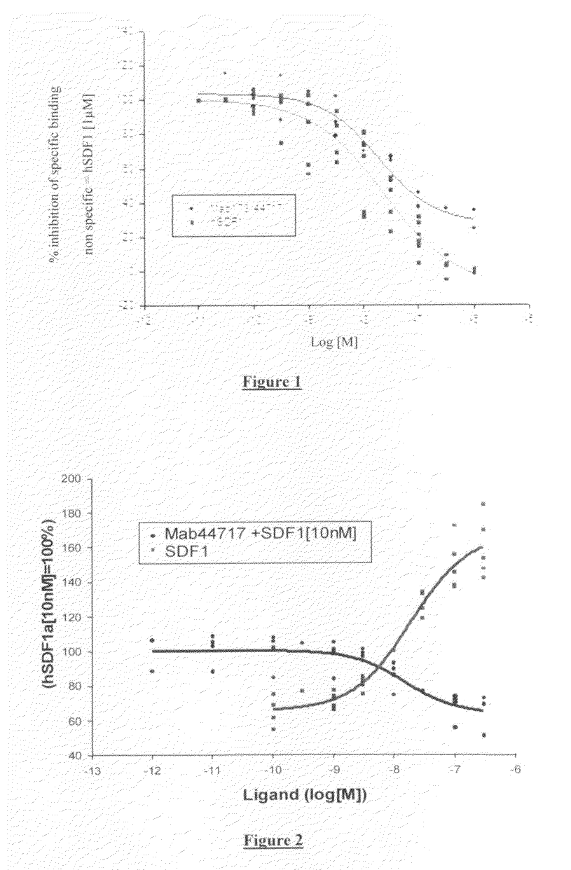

SDF1 stimulates the binding of [³⁵S]GTPγS in dose-dependent manner, reflecting activation of the CXCR4 receptor. The maximum stimulation obtained is 143% relative to the baseline binding of [³⁵S]GTPγS with a strength of 30.8±9.7 nM (n=16) (FIG. 2). Under these test conditions, the antibody 44717 shows maximum inhibition of stimulation of the binding of [³⁵S]GTPγS brought about by SDF1 (10 nM) of 58% at a dose of 300 nM and an IC₅₀ value of 20.7±5.9 nM (n=4) (FIG. 2).

### Example 3

### Test for Mobilisation of Intracellular Calcium Mediated by CXCR4

This test makes it possible to assess CXCR4 receptor signal transduction via the phospholipase C pathway, bringing about the release of intracellular calcium. This kinetic test makes it possible to monitor changes in the system over time.

Method:

CHO-K1 cells constitutively expressing the human CXCR4 receptor are obtained in the same manner as described in Example 1. The cells are seeded on black 96-well plates [100 000 cells in a medium of DMEM-F12—FCS 5%/well] and weaned overnight. The cells are loaded with fluorescent calcium probe (Fluo-4 No Wash) in the buffer [HBSS 1×, HEPS 20 mM, Probenicid acid 25 mM] for 30 min. at 37° C. and then 30 min. at 25° C. For the antagonism measurement, 10 μl of antibody 44717 solution are added to the cells. After incubating for 10 min. at ambient temperature, the measurement is carried out using the Mithras LB940 reader (Berthold) in fluorescence mode using the following parameters: excitation at 485 nm, emission at 535 nm and lamp at an energy of 10000. Each well is read for 0.1 second, every second over a period of 20 seconds (baseline signal) and then 20 μl of SDF-1 are injected and reading of the well is resumed for 2 min. Each test condition is tested in duplicate. The results are corrected by subtracting the values of a well without cells from the values of wells with cells. The values are then expressed in terms of % of the baseline signal and corrected so that the first value after addition of SDF-1 is 100%. This makes it possible to compensate for the indeterminate jump in fluorescence caused by injection of the ligand.

Results:

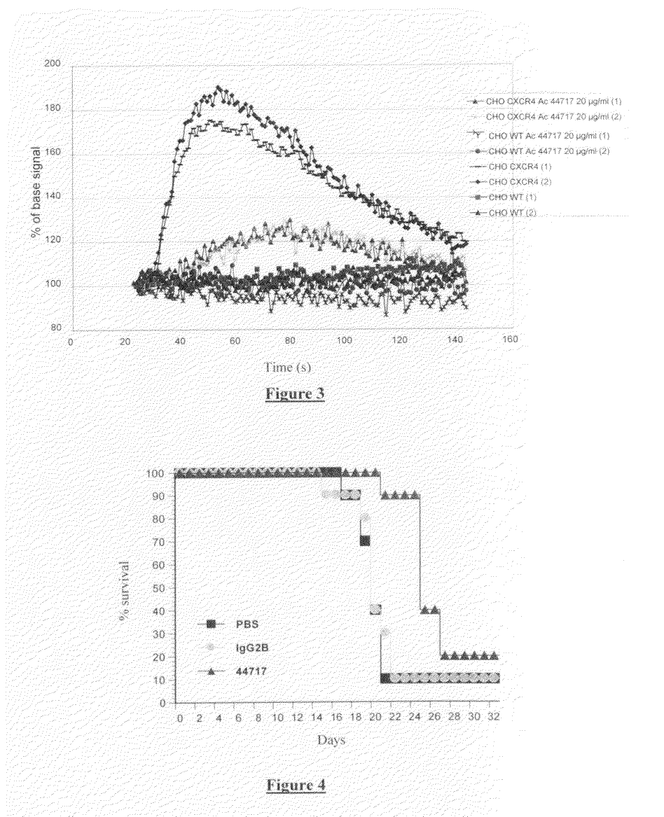

SDF1 (300 nM) causes release of calcium with the CHO/CXCR4 cells, whereas no response is observed in naïve CHO-K1 cells. The maximum signal is obtained after about 30 sec and the maximum effect of SDF-1 is 180% of the baseline signal (FIG. 3). The antibody 44717 (133 nM) greatly inhibits the SDF1-induced release of calcium, which reaches a maximum of 130% of the baseline signal after one minute following the injection of SDF1 (FIG. 3).

### Example 4

### Anti-Tumour Activity of the Antibody 44717 in the U937 Xenograft Model in the NOD Scid Mouse

Method:

U-937 cells were cultured in RPMI 1640 medium with phenol red and 4.5 g/l of glucose (Sigma, ref G8769), with added FCS 10% (F7524, Sigma) and L-Glutamine 2 mM (BioWhittaker ref BE17-605E). The cells were seeded two days before grafting; on implantation into the animal they were in the exponential growth phase. Ten million U-937 P((2)+4) cells were grafted into 8-week female Nod/Scid mice by intraperitoneal injection (strain: SOPF/NOD.CB17PRKDC/J FE 6 S, NOD.CB17-Prkdc scid/J, ref NSCSSFE06S, Charles River Laboratories).

Two days after grafting, the mice were treated (subcutaneous dorsal injections) according to the following schedules:
Control group: PBS, twice per week
IgG2b group: IgG2b, (Sigma ref M2695, Sigma batch 046K4843, batch 22060307, 3 mg/ml), 1 mg/dose per mouse, twice/week
44717 group: Anti-hCXCR4 monoclonal antibody (R&D Systems, ref MAB173, batch AUZ04512A, 3.43 mg/ml), 1 mg/dose per mouse, twice/week

The first dose was 2 mg per mouse for the control IgG2b antibodies and the antibody 44717. Selection of the s.c. route for treatment was made so as to avoid any direct contact between the tumour cells and the antibody in the peritoneal cavity.

Daily monitoring of the survival of the mice in each group was carried out.

Results:

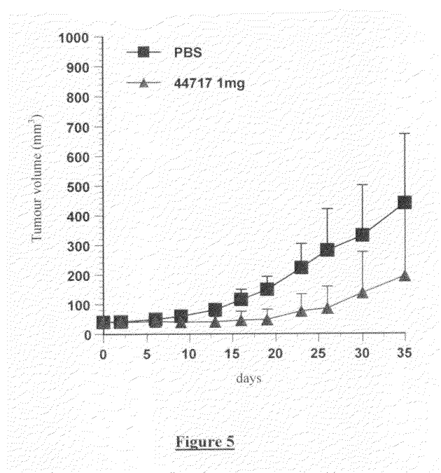

The first mouse died after 15 days in the IgG2b group. All the mice except one died in the following seven days (FIG. 4). The mouse which survived was euthanised 82 days after the graft and examined; there were no tumour cells in its abdominal cavity.

In the PBS group, all the mice except one died between 17 and 21 days after the graft (FIG. 4). The last died 82 days after the graft.

As regards the 44717 group, the mice started to die with a time lag of 4 to 6 days relative to the two other control groups. Eight mice died between days 22 and 27. Two mice survived to day 109, on which day they were euthanised (FIG. 4). Statistical analysis of the survival results (Kaplan-Meier) was carried out using the Log-Rank test (FIG. 4). The experiment described here shows that the monoclonal antibody 44717 directed to the CXCR4 receptor is capable of increasing the survival of mice in the U937 xenograft model.

### Example 5

### Inhibition of Tumour Growth by the Antibody 44717 in the MDA-MB-231 Xenograft Model in the Athymic Nude Mouse

Method:

MDA-MB-231 cells (ECACC) were cultured in DMEM medium (Invitrogen Corporation, Scotland, UK), in the presence of 10% FCS (Sigma). The cells were seeded 60 hours before the graft. On implantation they were in the exponential growth phase. Ten million MDA-MB-231 (P35+18) cells in PBS were grafted into 7-week Athymic Nude mice (HARLAN). Five days after implanting, the tumours could be measured (37 mm³<V³<44 mm³) and the animals were randomly divided into groups of 12 mice having tumours of comparable sizes. The mice were then treated by the i.p. route with a challenge dose of monoclonal antibody 44717 (batches AUZ05607A and AUZ04512A, R&D Systems) of 2 mg/mouse. Subsequently the mice were administered a dose of monoclonal antibody 44717 of 1 mg/mouse twice a week. In this experiment, a group of control mice was administered an equivalent volume of PBS.

The volume of the tumours was measured twice a week and calculated using the following formula: π/6×length×width×height.

A statistical analysis was carried out for each measurement using the Mann-Whitney test.

Results:

In the protocol 01MDAMB23101406, no mortality was observed during the treatment. Compared to the control PBS group, there is significant inhibition of tumour growth between days 6 and 35 (p≦0.05) for the monoclonal antibody 44717 administered at 1 mg/dose.

The mean tumour volume after 5 weeks of treatment was reduced by 56% for the antibody 44717 relative to the PBS.

The experiment described here shows that the monoclonal antibody 44717 directed to the CXCR4 receptor is capable of inhibiting tumour growth in a MDA-MB-231 xenograft model in the athymic Nude mouse.

1-13. (canceled)

14. A method for the treatment and/or prevention of cancer comprising administering to a human or animal subject, an effective amount of a monoclonal antibody, selected from the antibodies MAB170, MAB171, MAB172 and MAB173, and functional fragments thereof, wherein the monoclonal antibody, or a functional fragment thereof, is/are capable of specifically binding to the protein CXCR4 and of inhibiting in vitro and/or in vivo tumour growth of a primary tumour.

15. The method of claim 14, wherein the monoclonal antibody, or a functional fragment thereof, inhibits vascularisation in the primary tumour.

16. The method of claim 14, wherein the monoclonal antibody, or a functional fragment thereof, inhibits proliferation of tumour cells forming the primary tumour.

17. The method of claim 15, wherein the monoclonal antibody, or a functional fragment thereof, causes apoptosis of tumour cells forming the primary tumour.

18. The method of claim 14, wherein the monoclonal antibody consists essentially of the antibody MAB173.

19. The method of claim 14, wherein the cancer is selected from cancer of the colon, breast, prostate, lung, ovary, pancreas, kidney and brain, and lymphomas.

20. The method of claim 19, wherein the lung cancer is selected from small-cell lung cancer and non-small-cell lung cancer.

21. A composition comprising at least one active ingredient which is capable of inhibiting in vitro and/or in vivo tumour growth of a primary tumour, wherein the at least one active ingredient consists essentially of a monoclonal antibody, selected from the antibodies MAB170, MAB171, MAB172 and MAB173, and functional fragments thereof, which is/are capable of specifically binding to the protein CXCR4.

22. The composition of claim 21, wherein the anti-CXCR4 monoclonal antibody, or functional fragments thereof, consists essentially of the antibody MAB173.

23. The composition of claim 21, further comprising at least one pharmaceutically acceptable carrier.

24. The composition of claim 21, which is in the form of a combination therapy and is administered in a simultaneous, separate or time-staggered regimen with at least one cytotoxic/cytostatic agent and/or cell toxin and/or radioelement.

25. The composition of claim 21, further comprising at least one second anti-tumour antibody.

26. A method for the treatment of cancer comprising administering to a human or animal body, an effective amount of the composition of claim 21.

27. The method of claim 26, wherein the cancer is selected from cancer of the colon, breast, prostate, lung, ovary, pancreas, kidney and brain, and lymphomas.

28. The method of claim 27, wherein the lung cancer is selected from small-cell lung cancer and non-small-cell lung cancer.
